(19) United States
(12) Patent Application Publication
(10) Pub. No.: US 2018/0088080 A1
(43) Pub. Date: Mar. 29, 2018

(54) METHODS AND APPARATUS FOR MITIGATION OF CURRENT REVERSAL IN CAPILLARY ZONE ELECTROPHORESIS-ELECTROSPRAY DEVICE

(71) Applicant: UNIVERSITY OF NOTRE DAME DU LAC, South Bend, IN (US)

(72) Inventors: Norman J. Dovichi, South Bend, IN (US); David B. Go, South Bend, IN (US); Scott A. Sarver, South Bend, IN (US); Ryan J. Flaherty, Grand Rapids, MI (US); Gregory A. Brownell, South Bend, IN (US)

(21) Appl. No.: 15/716,149

(22) Filed: Sep. 26, 2017

Related U.S. Application Data
(60) Provisional application No. 62/400,036, filed on Sep. 26, 2016.

(51) Int. Cl. G01N 27/447; B01L 9/06; B01D 59/42; C07K 1/26

(52) U.S. Cl. CPC G01N 27/44743; G01N 27/44782; G01N 2035/00237; B01D 59/42; C07K 1/26; B01L 9/065

(57) ABSTRACT

The combined capillary electrophoresis electrospray mass spectrometry apparatus has a circuit to handle excess current allows separations under a wide range of electrophoretic conditions. The apparatus includes an electrospray with an emitter and an electrospray interface connected with a separation capillary configured to transport a sample with an injection end and a distal end. The injection end of the separation capillary is inserted into a reservoir containing a background electrolyte and the distal end is threaded within the electrospray interface and sized and shaped to mate with the electrospray interface. A power supply is electrically connected to the injection end and an amplifier at least one first diode positioned between the amplifier and the distal end allows current to flow to the distal end only. A second diode positioned between the distal end and a ground configured to allow current flow to the ground.

### CROSS REFERENCE TO RELATED APPLICATION

This application is a non-provisional claiming priority to U.S. Patent Application No. 62/400,036 entitled “CE Electrospray Distal-End Power Supply,” filed on Sep. 26, 2016, the contents of which are incorporated herein by reference in their entirety.

### GOVERNMENT LICENSE RIGHTS

This invention was made with government support under Contract No. R01GM096767 awarded by National Institutes of Health (“NIH”). The government has certain rights in the invention.

### FIELD OF THE DISCLOSURE

The present description relates generally to a current reversal mitigation and more particularly to methods and apparatus for mitigation of current reversal in capillary zone electrophoresis-electrospray devices.

### BACKGROUND OF RELATED ART

As depicted in FIG. 1A, a conventional CZE-ESI system consists of a separation capillary and two power supplies. The injection end of the capillary is placed in a reservoir containing the background electrolyte. The distal end of the capillary is threaded within the electrospray interface. A high-voltage power supply is connected to the injection end of the capillary. A second power supply controls the voltage at the distal end of the capillary. Connection between the second power supply and the distal end of the capillary is made either through a sheath fluid, through a thin segment of etched capillary, or through direct electrical connection. The mass spectrometer inlet is typically held at ground potential.

As shown in FIG. 1B, the electrical circuit can be drawn as three resistors, one corresponding to the separation capillary, a second corresponding to the transfer capillary between the second power supply and the electrospray interface, and a third corresponding to the electrospray. The resistance associated with the transfer capillary (R_{transfer}) tends to be quite low, and the potential applied to the electrospray interface is ideally very close to the potential supplied by HV2. However, conventional high voltage power supplies used for electrospray are sources of current but are unable to act as a current sink. Tens of microamperes of current can pass through the separation capillary, while the electrospray current leaving the sprayer is typically hundreds of nanoamperes. The power supply responsible for the spray voltage, (HV2) operates as a current source and the control circuit of that power supply holds the electrospray emitter at the desired voltage. However, when the capillary electrophoresis system is operated under relatively high current conditions, a situation can arise wherein the current flowing through the capillary is greater than the current generated by HV2. In this case, the electrospray voltage is not controlled by HV2 and instead floats to a higher value.

When the electrospray voltage is not accurately controlled as in the conventional devices, the sensitivity, reproducibility, and detection limit of the instrument suffer. To avoid the current-sinking challenge, separations are performed in low current conditions (i.e. <10 μA). When a large inner diameter capillary (e.g. 50 μm) is used in order to improve the loading capacity, a low separation voltage (typically 400 V/cm or lower) is necessary which limits the analysis throughput. When an improvement the analysis is tried throughput by increasing the separation voltage (e.g. 1,000 V/cm), it is often required to use a very small inner diameter separation capillary (e.g. 10 μm), which significantly limits the loading capacity, leading to low signal intensity and identification capacity. If the current-sinking challenge is mitigated, shorter capillaries with larger inner diameters may be employed while using higher conductivity separation buffers, which enable faster separations and larger loading amounts. Previous solutions to this problem float the capillary electrophoresis circuitry on the electrospray voltage which results in the effective capillary voltage no longer depending on the electrospray voltage. However, this first approach would increase the cost of the setup due to the need for additional safety precautions, such as isolated electronics on the capillary electrophoresis instrument.

### BRIEF DESCRIPTION OF THE DRAWINGS

FIG. 1A is a schematic diagram of a prior-art capillary electrophoresis-electrospray ionization-mass spectrometer apparatus.

FIG. 1B is a representative circuit of the device of FIG. 1A.

FIG. 2A is a schematic diagram of a sheathed capillary electrophoresis-electrospray ionization-mass spectrometer instrument apparatus according to the teachings of the present disclosure.

FIG. 2B is a schematic diagram of a sheathless capillary electrophoresis-electrospray ionization-mass spectrometer instrument apparatus according to the teachings of the present disclosure.

FIG. 2C is a circuit diagram of the protective circuit according to the teachings of the present disclosure.

FIG. 3 is a graph of the spray voltage measured separation voltage applied for 0.1% FA (a), 5% acetic acid (b), and 0.5% FA (c).

FIG. 4 is a graph of the base peak electropherograms of 0.5 mg/mL BSA digest comparing the present device (a) and a conventional power supply (b).

FIG. 5 is a graph of the electropherograms from 2 μM angiotensin II with the present device (a) and the conventional power supply (b).

FIG. 6 is a graph of the electropherograms of 2 μM angiotensin II with the HVM amplifier and a separation voltage of 19 kV (a), the SPELLMAN power supply and a separation voltage of 11 kV (b), and the SPELLMAN power supply and a separation voltage of 19 kV (c).

### DETAILED DESCRIPTION

The following description of example methods and apparatus is not intended to limit the scope of the description to the precise form or forms detailed herein. Instead the following description is intended to be illustrative so that others may follow its teachings.

Combined capillary-zone electrophoresis-electrospray ionization-mass spectrometry (CZE-ESI-MS) is attracting renewed interest in use for molecular analysis of many biomolecules and other samples. This interest is stimulated by the development of high-sensitivity interfaces for coupling CZE to MS.

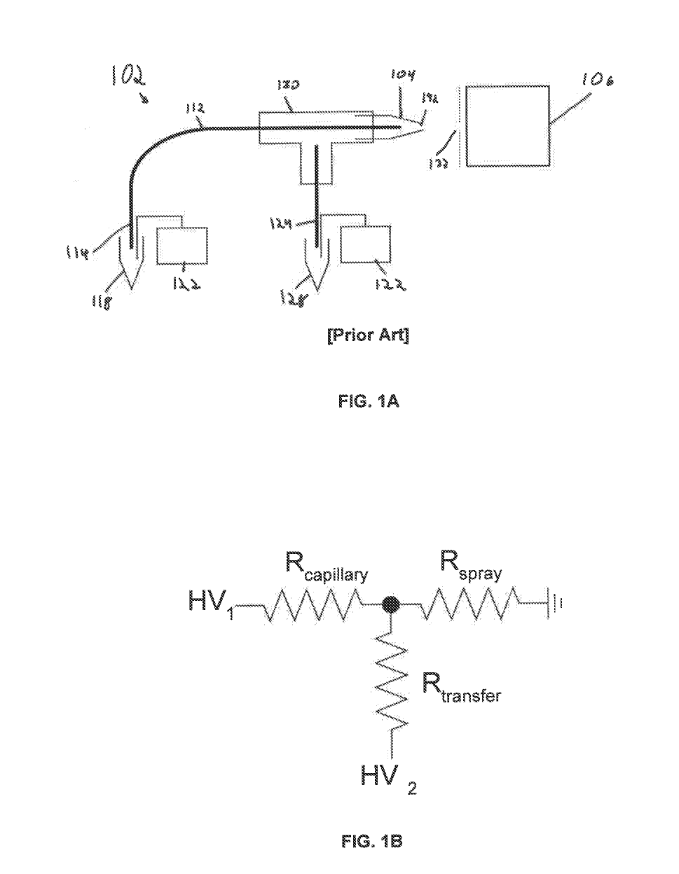

Referring now to FIG. 1A, an example of a prior art combined CZE-ESI-MS device for capillary-zone electrophoresis leading to electrospray ionization input into mass spectrometry for sample analysis. The combined device allows simultaneous separation, ionization, and identification of the sample. These devices are individually capillary electrophoresis (CE) instrumentation 102, electrospray 104, and mass spectrometer 106.

The convential capillary electrophoresis instrumentation 102 consists of a separation capillary 112 for transporting a sample with an injection end 114 and a distal end 116, the injection end 114 being inserted into a reservoir 118 containing a background electrolyte. The separation capillary 112 introduces the sample by capillary action, pressure, siphoning, or electrokinetically. A voltage differential is applied across the injection end 114 and the distal end 116, causing the sample to migrate across the capillary 102. A number of forms of capillary electrophoresis exist including capillary zone electrophoresis (CZE), but other electrophoretic techniques including capillary gel electrophoresis (CGE), capillary isoelectric focusing (CIEF), capillary isotachophoresis and micellar electrokinetic chromatography (MEKC). In each, separation occurs when electrokinetic motion outpaces osmotic motion, sorting the ions within the same by a combination of charge and weight.

In a device shown in FIG. 1A, two high voltage power supplies 122 are electrically connected to the separation capillary 112 and the transfer capillary 113. These electrical potential differences drive both the electrokinetic motion of the capillary electrophoresis apparatus 102 and electrospray 104. The sample is carried from the injection end 116 of the separation capillary 112 into the electrospray 104 where the sample is ionized, aerosolized out the emitter 142. As the mass spectrometer inlet 232 is typically held at a grounded potential, FIG. 1B simplifies the device as a simple circuit as discussed above.

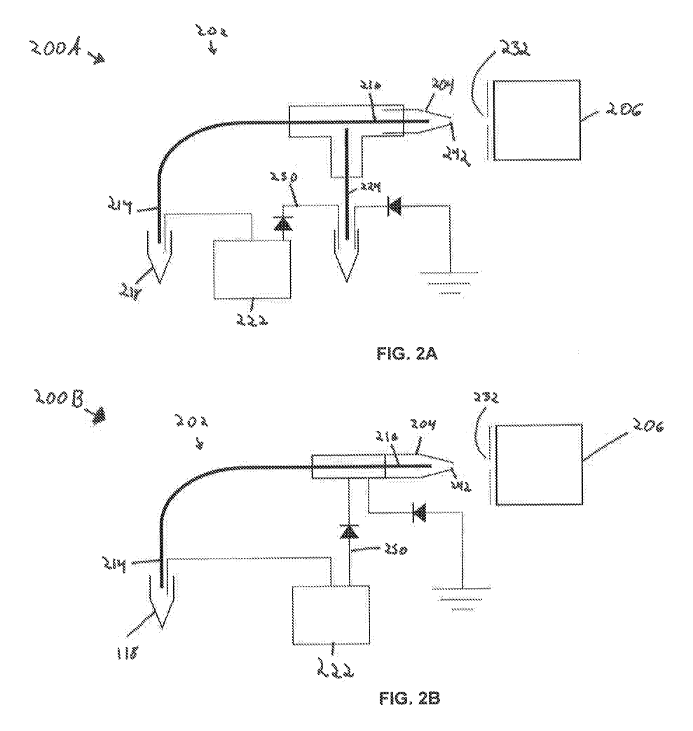

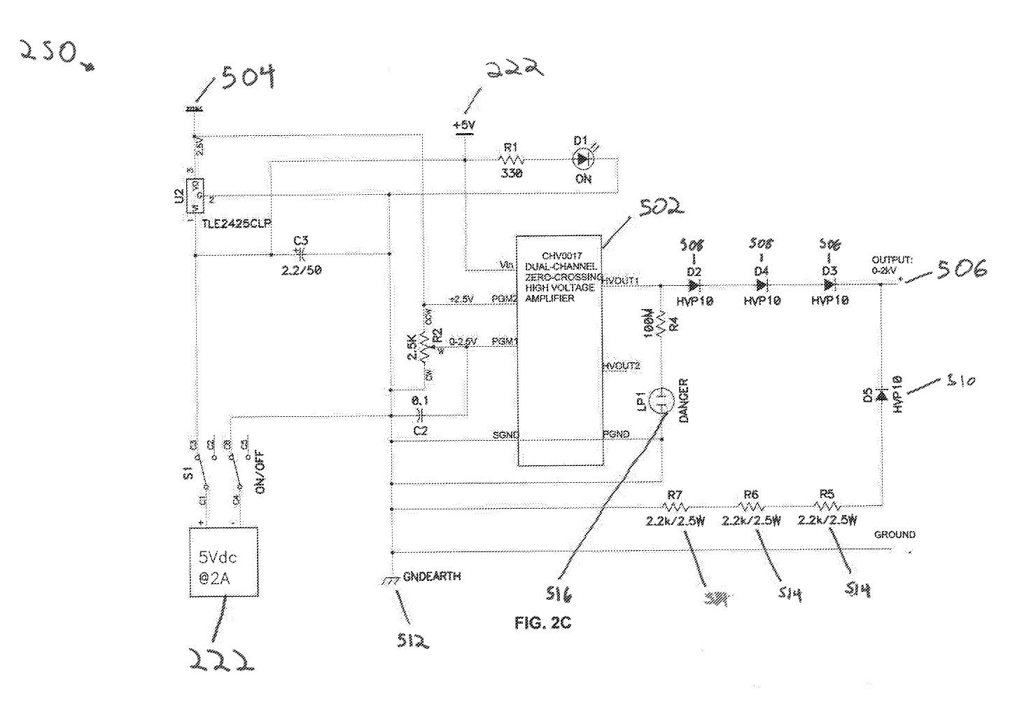

Referring now to FIGS. 2A-2C which show an implementation of a protective apparatus to protect the power supply from current backflow. FIGS. 2A-2B show two example devices of the present disclosure implementing the protective circuit. FIG. 2C shows a circuit diagram according to the teachings of the present disclosure to create the devices of FIGS. 2A and 2B for sample analysis at a wider range of electrophoretical conditions.

Compared to the prior art device shown in FIG. 1A which used two power supplies, the example devices shown in FIGS. 2A-2C use only a single power supply 202 connected to both outputs, one each at the capillary electrophoresis and electrospray inputs devices. Normally, in the prior art device, the second output would allow current to flow into the second connection at the electrospray and distal end of the capillary due to the electrical flow caused by the electrophoretic current. In this event, the current flow would also cause the target voltage to decouple from the exact output voltage of the power supply due to uncontrolled inputs of current. So in the examples shown, the circuit diagram of FIG. 2C shows a protection circuit to prevent current flow into the power supply 222 and shunt that unwanted flow to a ground.

FIG. 2A shows a protective circuit 250 implemented in a sheathed combined CZE-ESI-MS sheathed apparatus 200A. FIG. 2B shows the protective circuit in a sheathless configuration of the combined CZE-ESI-MS, sheathless apparatus 200B. Both devices 200A, 200B shown in FIGS. 2A-2B include a capillary electrophoresis (CE) instrumentation 202, an electrospray 204, and a mass spectrometer 206 similar to the capillary electrophoresis (CE) instrumentation 102, the electrospray 104, and the mass spectrometer 106 as explained above. Both devices shown in FIGS. 2A-2B include a separation capillary 212 with an injection end 214 and a distal end 216. The electrospray 204 is adapted to be received the distal end 216 of the separation capillary 212 and is located proximate to an inlet 232 on a mass spectrometer 206.

The separation capillary 212 is a submillimeter diameter tube, constructed of fused silica in the example shown, other materials for the capillary walls are contemplated such as polyamide walls. In some versions, like sheathless apparatus 200B, the capillary 212 is coated in an electrical conductor, called a sheathless design. In other versions, the capillary 212 is sheathed using a coaxial capillary 230 with a sheath liquid provided by a transfer capillary 224. Both the buffer liquid in the reservoir 218 and the sheath liquid in the transfer liquid reservoir 228 are made of a combination of a base and an additive. The base is water, methanol, or any other suitable material and the additive is acetic or formic acid or another suitable chemical. In some versions, a plastic or metal protective layer is used to protect these capillary layers.

In the combined devices of FIGS. 2A-2B, the capillary electrophoresis instrumentation 202 is sized and shaped to link with an electrospray 204. Then, the distal end 216 of the separation capillary 212 is threaded within an interface of the electrospray 204 to be press fit inside. This allows the separated components of the sample to be ionized and aerosolized without fragmentation before being analyzed by the mass spectrometer 206. The electrospray emitter 242 is located proximate to a mass spectrometry inlet 232 of the mass spectrometer 206 such that the sample is able to be received by the spectrometer 206.

In both sheathed apparatus 200A and sheathless apparatus 200B, the example device includes a single power supply 222 adapted with a protective circuit 250. In FIG. 2A, the protective circuit 250 is electrically connected to the separation capillary 212 and the transfer capillary 224 in reservoir 228. In FIG. 2B, the power supply is connected to the separation capillary 212 and the conductive element 220. The protection circuit 250 of FIG. 2C is also contemplated to work with two power supplies 222 while electrically isolating the current flow from each other.

Referring now to FIG. 2C, a circuit diagram shows the protective circuit 250, with the power supply 222 electrically connected to a first outlet 504 placed at the injection end 214 of the separation capillary, and an amplifier 502. The example power supply 222 comprises at least one first diode 208 positioned between the amplifier 502 and a second output 506 at the distal end 216 of the separation capillary 212 interfacing with the electrospray 204 configured to allow current to flow to the distal end of the separation capillary 212. More specifically, in the example shown, three diodes 508 are arranged sequentially, and are oriented to block any reverse current flow from the second output 210 at the electrospray 204. A second diode 510 or potentially series of diodes is positioned between the second output 506 and a ground 512 configured to allow current flow to the ground 512. In additional instances, the example protective circuit 250 may also include a series of resistors 514 or an electrical ballast (not shown) in line with the second diode 510 to control the rate of current flow in this discharge scenario.

The example electrospray power supply 202 with the protective circuit 250 sinks current when coupled with a diode-based protection circuit to handle situations where there is significant current reversal. In the following figures, an example system constructed in accordance with the teachings of the present invention, was subject to a number of test and the example system's capabilities were demonstrated and compared to a conventional configuration as illustrated in FIG. 1A using a SPELLMAN™ CZE-1000R, available from SPELLMAN High Voltage Electronics Corporation, Hauppauge, N.Y., and which is commonly used by researchers in the field.

For the results shown, Acetic acid and hydrofluoric acid (HF) were used in the base solution. Samples are of Bovine serum albumin (BSA), angiotensin II (human, Asp-Arg-Val-Tyr-Ile-His-Pro-Phe), and formic acid (FA) were used. In the tested example of the teachings of this disclosure the fused silica separation capillary 212 and the electrospray emitters 502 were constructed of borosilicate glass capillary. An LTQ XL™ Linear Ion Trap Mass Spectrometer, available from Thermo Fisher Scientific Inc., Waltham Mass. was used for all experiments. Only MS1 spectra were acquired. The scan range of the ion trap mass analyzer was m/z 350-1800. The separation power supply 122 was a SPELLMAN CZE-1000R, as noted above.

For the test, BSA in 100 mM NH4HCO3 (pH 8.0) containing 8 M urea was denatured at 37° C. for 30 min, followed by standard reduction and alkylation with DTT and IAA. After dilution with 100 mM NH4HCO3 (pH 8.0) to reduce the urea concentration below 2 M, protein digestion was performed at 37° C. with trypsin at a trypsin/protein ration of 1/30 (w/w) for 12 h. After acidification with FA, the protein digest was desalted with a C18-SepPak column and then was lyophilized with a vacuum concentrator. The dried protein digest was stored at −20° C. before use.

The example protection circuit 250 disclosed herein was designed and fabricated for the HVM amplifier to prevent current from passing into the HVM amplifier when excess current was generated during electrophoresis. As previously noted, a schematic of the HVM Technologies amplifier protection circuit 250 is illustrated in FIG. 2C In this demonstration, the protection circuit 250 comprised a series of rectifier diodes 508 such that when the electrophoresis was operated under high current conditions, the excess current that flowed toward the HVM amplifier was instead diverted to ground. In particular, a series of three diodes 508 (FIG. 2C) comprise the equivalent of an electrical check valve. That is, they permit a maximum positive current of 750 milliamps to be sourced from the HVM amplifier, while simultaneously blocking up to 30 kV from flowing back into the HVM. Thus, the arrangement of the protective circuit 250 allows both the separation and electrospray power supplies to source current to the CE-ESI circuit, while isolating the two supplies from each other. A fourth diode 510 protects the HVM amplifier from reverse current by shunting it directly to ground in the event that there is an excess of reverse current. It is noted that some reverse current can leak through the diodes (up to 5 μA through the D2-D3-D4 circuit made of diodes 508). The HVM is also reported to have some current sinking ability from the manufacturer, and this current sinking likely played the role of a secondary protection against this leakage current. Overall, this combination of HVM and protection circuit created a power supply that could mitigate potential current reversals, and limit spray voltage instabilities.

For initial power supply evaluations, a 60 cm bare fused silica capillary (50 μm i.d., 150 μm o.d.) was used to initially test the performance of the HVM amplifier and the SPELLMAN power supply. The distal end 216 of the separation capillary 212 was not etched by HF in this initial experiment. The example electrospray emitter 242 had an opening of 10 μm. The sheath buffer used was 0.1% (v/v) FA in water containing 10% (v/v) methanol. Three background electrolytes were used. The first separation buffer was 0.1% (v/v) FA, the second separation buffer was 5% (v/v) acetic acid, and the third separation buffer was 0.5% (v/v) FA.

The power supplies 122, 222 were controlled by LabVIEW software. The injection end of the separation capillary and an electrode were fixed in an injection block. The electrode provided high voltage for capillary electrophoresis separation. Nitrogen gas was used to provide pressure for capillary flushing and sample injection; no pressure was used during separation.

A constant spray voltage (1.4 kV) was used for these experiments. The separation voltage applied at the injection block was increased from 1 to 30 kV in 2 kV increments. This procedure was performed for the background electrolytes listed above. Spray voltage was measured in the vial that supplied the sheath buffer using a Fluke 80K-40 HV Probe. Prior to applying the separation voltage, the spray voltage was set and measured with the high voltage probe.

A second series of experiments used a 31 cm long, 20-μm ID, 150 μm OD separation capillary. Approximately 5 mm of the distal end of the separation capillary was etched using HF to an o.d. of ˜45 as reported earlier. The etched capillary allowed placing the distal end of the capillary a few micrometers from the emitter opening, which increased sensitivity. The electrospray emitter used in this experiment had an opening of 20 μm. The sheath electrolyte used in the electrospray emitter was 0.1% (v/v) FA in water containing 10% (v/v) methanol. The background electrolyte was 5% (v/v) acetic acid. The separation voltage was 27 kV and the spray voltage was 1.6 kV for all experiments with the 20 μm i.d. capillary.

The length of the injection plug was estimated using:

$$L = \frac{P \times S \times {D}^{2}}{3200 \times C} , \qquad ( 1 )$$

where L is the injection length in mm, P is the pressure in mbar (1 mbar≈0.015 psi), S is the injection time in seconds, C is the capillary length in cm, and D is the capillary inner diameter in μm. The injection conditions used were 2.0 seconds at 10 psi, which gave an injection length of 5.4 mm. The first sample used was a BSA digest diluted in 0.1% (v/v) FA to a concentration of 0.5 mg/mL. The second sample used was angiotensin II diluted in 0.1% (v/v) FA to concentrations of 2, 5, 10, and 20 μM. Data were collected in triplicate.

A final set of experiments used a 31-cm long, 50-μm ID, 150 μm OD separation capillary. Approximately 10 mm of the distal end of the capillary was etched using HF to an o.d. of ˜65 The electrospray emitter, sheath buffer, and separation buffer were identical to those used in the 20 μm i.d. capillary experiment.

For this set of experiments, the spray voltage was increased to 1.7 kV. Three experimental conditions were employed, Table 1. The first condition used the HVM amplifier with a separation voltage of 19 kV. The second condition used the SPELLMAN power supply with a separation voltage of 11 kV. These first two conditions demonstrate the capabilities of each of the electrospray power supplies at the maximum voltage applied by the separation power supply that would not alter the spray voltage. The third condition used the SPELLMAN power supply with a separation voltage of 19 kV and provides a comparison of the ability of the two electrospray power supplies to mitigate current reversal.

TABLE 1
Separation and Electrospray Voltages for 50 μm i.d. Capillary

| | Separation | Spray |
|---|---|---|
| Electrospray Power | Voltage | Voltage |
| Supply | (kV) | (kV) |
| HVM Amplifier | 19 | 1.7 |
| SPELLMAN Power | 11 | 1.7 |
| Supply | | |
| SPELLMAN Power | 19 | 1.7 |
| Supply | | |

Injection conditions were calculated using Equation 1 to maintain a constant injection length of 5.4 mm. The injection conditions used were 0.8 seconds at 4 psi. The samples used for the 50 μm i.d. capillary experiment were identical to those used for the 20 μm i.d. capillary experiment. The first sample used was a BSA digest diluted in 0.1% (v/v) FA to a concentration of 0.5 mg/mL. The second sample used was angiotensin II diluted in

A 60-cm long, 50-μm ID separation capillary was used for the initial experiments. The performance of the HVM amplifier and SPELLMAN CZE-1000 power supplies were compared for three background electrolytes 0.1% (v/v) FA, 5% (v/v) acetic acid, and 0.5% (v/v) FA.xxxx The sheath electrolyte was 0.1% (v/v) FA in water containing 10% (v/v) methanol. A spray voltage of 1.4 kV was applied to the sheath electrolyte reservoir and monitored using a high voltage probe.

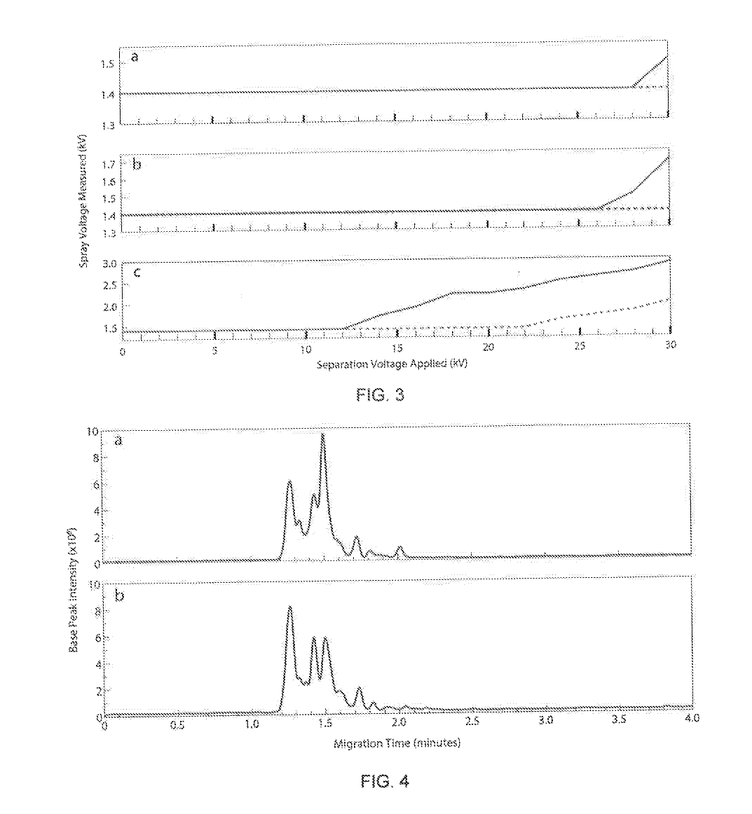

FIG. 3 presents the observed voltage in the sheath electrolyte reservoir as the separation voltage was increased. A positive deviation from 1.4 kV indicated that the electrospray power supply (HV [symbol] 2) failed to maintain the spray voltage due to the current passing through the separation capillary 0.0.1% (v/v) FA to concentrations of 2, 5, 10, and 20 μM. Data were collected in triplicate. In FIG. 3, the solid (blue) trace corresponds to the SPELLMAN power supply and the dashed (red) trace corresponds to the HVM amplifier. Approximate conductivities of electrolytes: 0.1% formic acid ˜0.3 mS/cm; 5% acetic acid ˜1.2 mS/cm; and 0.5% formic acid ˜5.6 mS/cm.

The HVM amplifier (red, dashed line) maintains the spray voltage at 1.4 kV while the separation voltage is ramped to 30 kV with both 0.1% FA and 5% acetic acid background electrolytes. Only when the highest conductivity buffer is used, 0.5% FA, does the HVM amplifier fail to maintain the 1.4 kV spray voltage at the highest separation voltages. In contrast, the SPELLMAN power supply (solid, blue line) is unable to maintain the set electrospray voltage at the highest separation voltage for all three background electrolytes, and the separation voltage that produces a deviation from the set voltage tracks the electrolyte conductivity.

Following the initial evaluation, the electropherograms were compared and generated using the HMV amplifier and SPELLMAN power supply with a separation voltage of 27 kV and spray voltage of 1.6 kV with the 20 μm i.d. capillary. The purpose of this experiment was to determine if the HVM amplifier could produce comparable capillary electrophoresis data to the SPELLMAN power supply under conditions with relatively low electrophoretic separation current.

The first sample that was tested was a BSA digest at a concentration of 0.5 mg/mL. The base peak electropherograms for the HVM amplifier and SPELLMAN power supply are shown in FIG. 4. The data from each electropherogram was smoothed with a 5-point Gaussian convolution. While the base peaks for the two power supplies differ, the overall profile of the BSA digest electropherograms is similar.

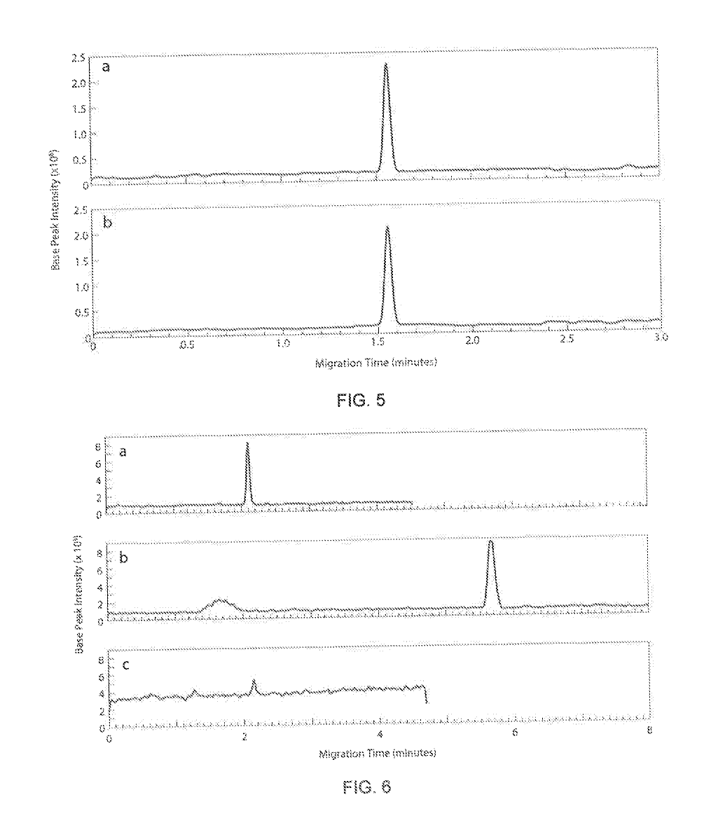

The next sample was angiotensin II at concentrations of 20, 10, 5, and 2 μM. Electropherograms of 2 μM angiotensin II are presented in FIG. 4 for the HVM amplifier (FIG. 5a) and the SPELLMAN power supply (FIG. 5b). The data from each electropherogram were smoothed with a 5-point Gaussian convolution. The peak intensities and migration times are very similar between the two power supplies.

Unweighted least squares plots were linear for both the HVM amplifier (slope=1.799×10⁶, R=0.9920) and the SPELLMAN power supply (slope=2.088×10⁶, R=0.9940). The y-intercepts for the HVM amplifier and the SPELLMAN power supply were equal to zero within experimental error. Thus, in general, the two power supplies both produced similar quantitative CE-ESI-MS performance.

A more stringent evaluation of the electrospray power supplies, the experiment was repeated with a 50 μm i.d. capillary. The increased inner diameter of the separation capillary provides a ˜six-fold increase in capillary cross-section, which results in a proportional increase in current flow through the separation capillary.

In the initial test of the 50 μm i.d. capillary, the SPELLMAN power supply was not able to mitigate spray voltage instability above a separation voltage of 11 kV, causing the spray voltage to deviate from its setting. The difference between the HVM amplifier and the SPELLMAN power supply is illustrated most dramatically when comparing the electropherograms from the 2 μM angiotensin II runs at the three voltage conditions in this experiment (FIGS. 6A-6C), which correspond to the three cases in Table 1.

The electropherogram generated by the HVM amplifier and a separation voltage of 19 kV (FIG. 6A) results in a migration time just over two minutes and a base peak intensity of roughly 800,000. The electropherogram generated by the SPELLMAN power supply and a separation voltage of 11 kV (FIG. 6B) results in a significantly later migration time (˜six minutes) with similar base peak intensity. The electropherogram generated by the SPELLMAN power supply and a separation voltage of 19 kV (FIG. 6c) results in a higher background and a lower base peak intensity of approximately 525,000. In FIG. 6C, the migration time of angiotensin II is identical to that of FIG. 6A where the HVM amplifier was used under exactly the same conditions. However, the base peak intensity in FIG. 6A is approximately 50% greater and the background is roughly one third of what is seen in FIG. 6C.

Again, unweighted least squares calibration curves were linear for the HVM amplifier (slope=1.4×10⁶, R=0.9945), the SPELLMAN power supply with a separation voltage of 11 kV (slope=1.5×10⁶, R=0.9962), and the SPELLMAN power supply with a separation voltage of 19 kV (slope=6.12×10⁵, R=0.9967). The y-intercepts for all three plots were equal to zero within experimental error.

Finally, SPELLMAN CZE-1000R power supplies were used widely for capillary electrophoresis separations and have been used extensively as an electrospray power supply. However, as illustrated here, the SPELLMAN power supplies are not well suited for fast separations that employ high electric fields with high ionic strength separation buffers. The protection circuit 250 of the example shown developed is clearly capable of mitigating spray voltage instability and would protect the amplifier from current reversal. In the event that some current passed into the HVM amplifier, its manufactured ability to sink current could offer a secondary protection to the integrity of the spray voltage. The limit was identified of where the HVM amplifier protection circuit failed to maintain the applied spray voltage (FIG. 3C). Overall, the HVM amplifier and the associated protection circuit 250 are well suited for fast separations that employ high electric fields and high ionic strength separation buffers.

The description detailed herein is described in reference to the particular testing apparatus to end at a mass spectrometer. It is contemplated that the protective circuit 250 described herein could be adapted to a series of capillary electrophoresis or other reactions in which continuous voltage control is important, but stray current flows may damage a power supply.

Although certain example methods and apparatus have been described herein, the scope of coverage of this patent is not limited thereto. On the contrary, this patent covers all methods, apparatus, and articles of manufacture fairly falling within the scope of the appended claims either literally or under the doctrine of equivalents.

We claim:

1. An apparatus for sample analysis at a wide range of electrophoretical conditions comprising:
an electrospray with an emitter and an electrospray interface;
a separation capillary configured to transport a sample with an injection end and a distal end, wherein the injection end of the separation capillary is inserted into a reservoir containing a background electrolyte and the distal end is threaded within the electrospray interface and sized and shaped to mate with the interface;
a first power supply electrically connected to the injection end and an amplifier;
at least one first diode positioned between the amplifier and the distal end configured to allow current to flow to the distal end; and
a second diode positioned between the distal end and a ground configured to allow current flow to the ground.

2. The apparatus of claim 1, wherein the electrospray further comprises an emitter which is located proximate to a mass spectrometry inlet.

3. The apparatus of claim 1, wherein the separation capillary is configured to separate the sample by one of the following: capillary zone electrophoresis, capillary gel electrophoresis, capillary isoelectric focusing, capillary isotachophoresis or micellar electrokinetic chromatography.

4. The apparatus of claim 1, wherein amplifier is connected to a sheath comprising a transfer capillary with a transfer electrolyte supplied from a transfer reservoir.

5. The apparatus of claim 1, wherein amplifier is connected to a conductive element located concentrically around the distal end of the separation capillary.

6. The apparatus of claim 2, wherein the voltage potential between the injector end and the distal end is between 11 kV and 19 kV.

7. The apparatus of claim 2, wherein the voltage potential between the injector end and the distal end is greater than 19 kV.

8. The apparatus of claim 1, wherein the diodes are rectifier diodes.

9. The apparatus of claim 1, further comprising a LED light.

10. The apparatus of claim 1, further comprising at least one additional power supply wherein the first power supply and the at least one additional power supply are electrically isolated from each other respectively.

11. The apparatus of claim 1, further comprising a resistor positioned between the second diode and the ground, whereby the resistor controls the current flow in a current reversal scenario.

12. An apparatus for sample analysis at a wide range of electrophoretical conditions comprising:
a separation means for transporting a sample, comprising an output;
an ionization means for ionizing the sample connected to the output of the separation means at an interface, further comprising an emitter;
a means for analyzing the sample located proximate to the emitter of the ionization; and
a protective means for preventing current from flowing into a power supply, the power supply operably connected to the separation means and the ionization means to provide a voltage differential.
